(19) United States
(12) Patent Application Publication
(10) Pub. No.: US 2021/0043292 A1
(43) Pub. Date: Feb. 11, 2021

(54) TECHNIQUES FOR PROVIDING THERAPEUTIC TREATMENT INFORMATION FOR PHARMACOLOGICAL ADMINISTRATION

(71) Applicant: RxAssurance Corporation (d/b/a OpiSafe), Denver, CO (US)

(72) Inventors: Robert J. VALUCK, Denver, CO (US); Thomas C. ENNIS, Denver, CO (US); Gary Wayne CLARK, Denver, CO (US)

(21) Appl. No.: 16/985,839

(22) Filed: Aug. 5, 2020

Related U.S. Application Data
(60) Provisional application No. 62/882,884, filed on Aug. 5, 2019.
(60) Provisional application No. 63/027,529, filed on May 20, 2020.

(51) Int. Cl. G16H 20/10; G16H 10/60; G16H 50/20; G06N 20/20; G06F 9/54

(52) U.S. Cl. CPC G16H 20/10; G16H 10/60; G06F 9/547; G06N 20/20; G16H 50/20

(57) ABSTRACT

Examples described herein generally relate to recommending therapeutic treatment information for a patient, and more particularly to recommendations regarding drugs or drug combinations. A computer system may access a plurality of data records associated with the patient. The system may correlate and consolidate the plurality of records based on an accuracy rating of each data source. The system may provide access, via a first application programming interface (API) to a first plurality of shared machine learning algorithms to perform a respective analysis of the plurality of data records. The system may receive, via a second API, a proprietary set of executable code to perform an analysis of the plurality of data records. The system may execute the first plurality of shared machine learning algorithms and the set of executable code on the plurality of data records for the patient to generate therapeutic treatment information for the patient.

This application claims priority to U.S. Provisional Application No. 62/882,884 titled “TECHNIQUES FOR PROVIDING THERAPEUTIC TREATMENT INFORMATION FOR PHARMACOLOGICAL ADMINISTRATION,” filed Aug. 5, 2019, and U.S. Provisional Application No. 63/027,529 titled “TECHNIQUES FOR PROVIDING THERAPEUTIC TREATMENT INFORMATION FOR PHARMACOLOGICAL ADMINISTRATION,” filed May 20, 2020, both of which are assigned to the assignee hereof, and incorporated herein by reference in its entirety.

### BACKGROUND

The present disclosure relates to decision support for doctors, and particularly to recommendations for therapeutic treatment protocols for pharmacological administration.

70,237 drug overdose deaths occurred in the United States in 2017. The age-adjusted rate of overdose deaths increased significantly by 9.6% from 2016 (19.8 per 100,000) to 2017 (21.7 per 100,000). Opioids—mainly synthetic opioids (other than methadone)—are currently the main driver of drug overdose deaths. Opioids were involved in 47,600 overdose deaths in 2017 (67.8% of all drug overdose deaths). Addiction to opioids (clinically referred to as Opioid Use Disorder, or OUD) is more common than previously thought, and affects between 1-5% of the U.S. population. Opioid prescribing comprises 9% of all prescribing in the U.S., and most individuals who become addicted or overdose began with prescribed opioids.

Current methods of discovering drug regimens for patients involve retrospective analysis of drug usage, if available, and arbitrary prescribing guidelines such as step therapy, based on limited information. For example, many medical providers use historical claims data in an attempt to detect individuals with suboptimal pain control, side effects, and/or potential opioid use disorder, and then intervene to implement more effective treatment plans. The current method of decision support for optimizing drug regimens is not effective as it lacks the critical knowledge impact of numerous relevant data sources, direct patient interaction, and systematic monitoring of patient data on an ongoing basis.

Thus, there is a need in the art for improvements in decision support for pharmacological administration. In particular, there is a need for systems and methods for evaluating possible therapeutic treatment protocols involving potentially addictive drugs.

### SUMMARY

The following presents a simplified summary of one or more implementations of the present disclosure in order to provide a basic understanding of such implementations. This summary is not an extensive overview of all contemplated implementations, and is intended to neither identify key or critical elements of all implementations nor delineate the scope of any or all implementations. Its sole purpose is to present some concepts of one or more implementations of the present disclosure in a simplified form as a prelude to the more detailed description that is presented later.

In an example, the disclosure provides a method of recommending therapeutic treatment information for a patient. The method may include accessing a plurality of data records associated with the patient. The method may include correlating and consolidating the plurality of records based on an accuracy rating of each data source. The method may include providing access, via a first application programming interface (API) to a first plurality of shared machine learning algorithms to perform a respective analysis of the plurality of data records. The method may include receiving, via a second API, a proprietary set of executable code to perform an analysis of the plurality of data records. The method may include executing the first plurality of shared machine learning algorithms and the set of executable code on the plurality of data records for the patient to generate therapeutic treatment information for the patient. The method may include providing the therapeutic treatment information for the patient.

In another aspect, the disclosure provides a system for recommending therapeutic treatment information for a patient. The system may include a memory storing computer-executable instructions and a processor configured to execute the computer-executable instructions. The processor may access a plurality of data records associated with the patient. The processor may correlate and consolidate the plurality of records based on an accuracy rate of each data source. The processor may provide access, via a first API to a first plurality of shared machine learning algorithms to perform a respective analysis of the plurality of data records. The processor may receive, via a second API, a proprietary set of executable code to perform an analysis of the plurality of data records. The processor may execute the first plurality of shared machine learning algorithms and the set of executable code on the plurality of data records for the patient to generate therapeutic treatment information for the patient. The processor may provide the therapeutic treatment information for the patient.

In another aspect, the disclosure provides a method of generating therapeutic recommendations and guidance for administration of drugs and drug combinations. The method may include receiving, via a patient interface, patient preferences for drug efficacy, side effect levels, and addiction risk for a patient with a condition being treated with a current drug. The method may include generating, based on the patient preferences, a weight for each of drug efficacy, side effect levels, and addiction risk. The method may include analyzing, using one or more trained machine learning algorithms, a plurality of data records to determine a score for drug efficacy, side effect levels, and addiction risk for each of a plurality of alternative drugs. The method may include providing a therapeutic success rating for the current drug and each of the plurality of alternative drugs based on the weight and the scores for the respective drug.

In another aspect, the disclosure provides a system for generating therapeutic recommendations and guidance for administration of drugs and drug combinations. The system may include a memory storing computer-executable instructions and a processor configured to execute the computer-executable instructions. The processor may receive, via a patient interface, patient preferences for drug efficacy, side effect levels, and addiction risk for a patient with a condition being treated with a current drug. The processor may generate, based on the patient preferences, a weight for each of drug efficacy, side effect levels, and addiction risk. The processor may analyze, using one or more trained machine learning algorithms, a plurality of data records to determine a score for drug efficacy, side effect levels, and addiction risk for each of a plurality of alternative drugs. The processor may provide a therapeutic success rating for the current drug and each of the plurality of alternative drugs based on the weight and the scores for the respective drug.

Additional advantages and novel features relating to implementations of the present disclosure will be set forth in part in the description that follows, and in part will become more apparent to those skilled in the art upon examination of the following or upon learning by practice thereof.

### DESCRIPTION OF THE FIGURES

In the drawings:

FIG. 1 is a diagram of an example computer system for providing therapeutic treatment information for pharmacological administration, in accordance with an implementation of the present disclosure.

FIG. 2 is a diagram illustrating an example system architecture, in accordance with an implementation of the present disclosure.

FIG. 3 is a diagram illustrating an example architecture for a machine learning component, in accordance with an implementation of the present disclosure.

FIG. 4 is a conceptual diagram of an example implementation of a computer system, in accordance with an implementation of the present disclosure.

FIG. 5 illustrates example records processing operations, in accordance with an implementation of the present disclosure.

FIG. 6 is a flowchart of an example method of providing therapeutic treatment information for pharmacological administration, in accordance with an implementation of the present disclosure.

FIG. 7 is a flowchart of an example method of providing therapeutic success ratings, in accordance with an implementation of the present disclosure.

FIG. 8 is a schematic block diagram of an example computer device, in accordance with an implementation of the present disclosure.

FIG. 9 is a diagram illustrating an example communications architecture in accordance with an implementation of the present disclosure.

FIG. 10 is a diagram illustrating an example application programming interface (API) structure, in accordance with an implementation of the present disclosure.

FIG. 11 is a diagram illustrating an example graphical user interface (GUI) for presenting therapeutic recommendations, in accordance with an implementation of the present disclosure.

FIG. 12 is a diagram illustrating an example GUI for presenting drug information, in accordance with an implementation of the present disclosure.

FIG. 13 is a diagram illustrating an example GUI for presenting patient risk, in accordance with an implementation of the present disclosure.

### DETAILED DESCRIPTION

The present disclosure provides systems and methods for providing therapeutic treatment information for pharmacological administration. The disclosure provides techniques that allow a computer system provider to support a health care provider in making decisions regarding prescribing drugs or drug combinations for a patient.

In an aspect, the systems may utilize multiple learning algorithms to analyze collected data records. The system may provide access to a first set of shared algorithms via a first application programming interface (API). The system may provide access to a second set of proprietary algorithms via a second API. Accordingly, a provider may execute a combination of shared and proprietary algorithms on the collected data records. The results of the algorithms may provide therapeutic treatment information, which may include a treatment protocol, educational information, pricing information, and/or medication information.

In another aspect, the system may include a patient interface that receives patient preferences regarding factors including management efficacy, side effect levels, and addiction risk. The system may generate weights for each factor based on the patient preferences. The system may generate a score for each factor using the machine learning algorithms applied to collected data for the patient. The system may provide a therapeutic success rating for a current drug and each of a plurality of alternative drugs based on the weight and scores for the respective drug.

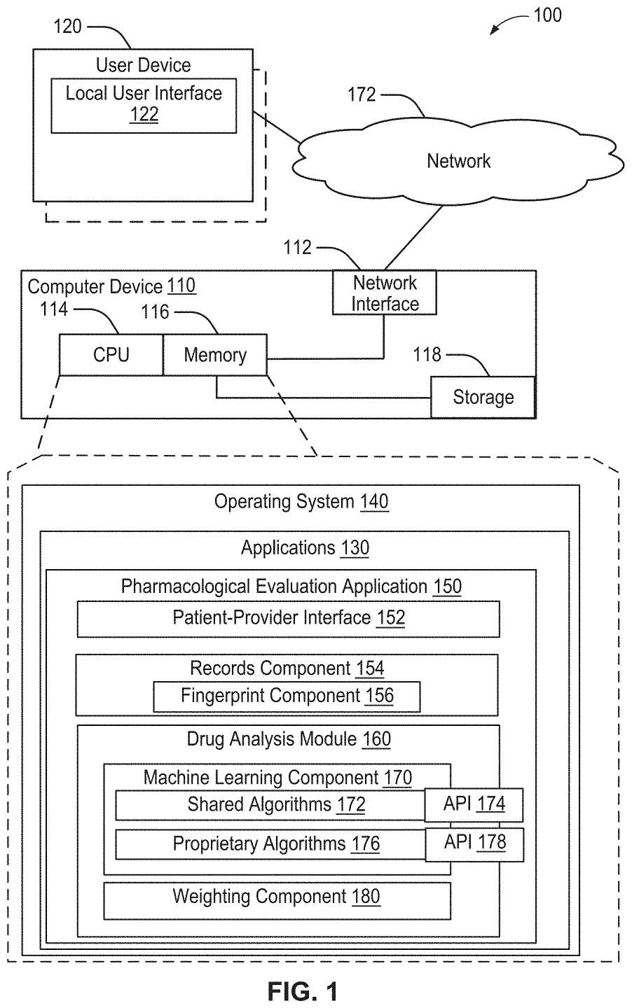

Referring now to FIG. 1, an example pharmacological evaluation system 100 includes a central computer device 110 and a plurality of user devices 120. The central computer device 110 may be, for example, any mobile or fixed computer device including but not limited to a computer server, desktop or laptop or tablet computer, a cellular telephone, a personal digital assistant (PDA), a handheld device, any other computer device having wired and/or wireless connection capability with one or more other devices, or any other type of computerized device capable of processing pharmacological related data. In an aspect, the central computer device 110 may be implemented as one or more virtual machines hosted by a web services provider.

In an aspect, the pharmacological evaluation system 100 may include a pharmacological evaluation application 150 executed by the computer device 110. The pharmacological evaluation system 100 may recommend a therapeutic treatment information for a patient and/or generate predictive probabilities of therapeutic and adverse outcomes with recommended patient treatment options for administration of drugs and drug combinations. The pharmacological evaluation application 150 may include a patient-provider interface 152 that receives input from a patient or health care provider, a records component 154 that accesses a plurality of data records associated with the patient; and a drug analysis module 160 that analyses the plurality of data records using one or more machine-learning algorithms. In an aspect, the machine-learning algorithms may include shared algorithms 172 and proprietary algorithms 176. A first application programming interface (API) 174 may provide access to the shared algorithms 172. A second API 178 may provide access to the proprietary algorithms 176.

The computer device 110 may include a central processing unit (CPU) 114 that executes instructions stored in memory 116. For example, the CPU 114 may execute an operating system 140 and one or more applications 130, which may include a pharmacological evaluation application 150. The computer device 110 may also include a network interface 112 for communication with external devices via a network. For example, the computer device 110 may communicate with a plurality of user devices 120.

Memory 116 may be configured for storing data and/or computer-executable instructions defining and/or associated with an operating system 140 and/or application 130, and CPU 114 may execute operating system 140 and/or application 130. Memory 116 may represent one or more hardware memory devices accessible to computer device 110. An example of memory 116 can include, but is not limited to, a type of memory usable by a computer, such as random access memory (RAM), read only memory (ROM), tapes, magnetic discs, optical discs, volatile memory, non-volatile memory, and any combination thereof. Memory 116 may store local versions of applications being executed by CPU 114. In an implementation, the memory 116 may include or communicate with a storage device 118, which may be a non-volatile memory.

In an aspect, the storage device 118 may include a blockchain storage. The blockchain storage may store immutable records by allowing append operations only such that the records are sequenced. Further, the records may use hash chaining such that each record may be cryptographically verified to provide data security. In an implementation, the blockchain storage may be distributed or duplicated across devices. In an aspect, the blockchain storage may be utilized for smart contracts. For example, a patient consent may be stored in the blockchain storage and verified when records for the patient are accessed.

The CPU 114 may include one or more processors for executing instructions. An example of CPU 114 can include, but is not limited to, any processor specially programmed as described herein, including a controller, microcontroller, application specific integrated circuit (ASIC), field programmable gate array (FPGA), system on chip (SoC), or other programmable logic or state machine. The CPU 114 may include other processing components such as an arithmetic logic unit (ALU), registers, and a control unit. The CPU 114 may include multiple cores and may be able to process different sets of instructions and/or data concurrently using the multiple cores to execute multiple threads. In an aspect, a graphics processing unit (GPU) may perform some operations of the CPU 114. For example, for blockchain operations, a GPU may be utilized for mining blocks (e.g., finding hash keys).

The operating system 140 may include instructions (such as applications 130) stored in memory 116 and executable by the CPU 114. The applications 130 may include a pharmacological evaluation application 150 configured to evaluate a set of collected data for a patient having a condition and provide therapeutic treatment information. The pharmacological evaluation application 150 may include a patient-provider interface 152 that may be in communication with or otherwise operate in conjunction with a local user interface 122 on a user device 120. The patient-provider interface 152 may be any user interface with which an end user may interact. For example, the patient-provider interface 152 may be an application or operating system that runs on the user devices 120. The pharmacological evaluation application 150 may be associated or in communication with an online store or update service. Accordingly, the pharmacological evaluation application 150 may occasionally publish an updated version of the patient-provider local user interface 122. As another example, the patient-provider interface 152 may be a web-page that is accessed through a browser application executed on the user devices 120. By loading the web-page, the browser application may effectively operate as a user interface for an application executed on the computer device 110 (e.g., in the case of a web server).

In an aspect, the patient-provider interface 152 may acquire patient preferences of a patient. For example, the patient-provider interface 152 may generate a survey or questionnaire that may be completed directly by a patient operating the user device 120, or may be completed by a medical provider based on answers provided by the patient. The patient preferences may include a relative ranking of management efficacy, side effect levels, and addiction risk. The patient preferences may include preferences regarding specific side effects. For example, the patient-provider interface 152 may generate questions regarding specific side effects based on a condition of the patient and/or possible drug recommendations. In another example, the patient-provider interface 152 may collect patient data that may be accessed and recorded from a wearable. The patient data may include biometric vital signs, including, but not limited to heart rate, heart rate variability, blood pressure, respiration, and temperature. Such patient data may be used to calculate or update clinically diagnosed conditions including, but not limited to pain, function, anxiety, depression, and sleep.

The pharmacological evaluation application 150 may include a records component 154. The records component 154 may access a plurality of data records for a patient, provider, payer, or drug. The records component 154 may correlate and rectifying the records based on an accuracy rating of each data source. For example, an electronic health record (EHR) may be considered a highest level of truth, but may be supplemented with information from other sources such as a state prescription drug monitoring program (PDMP), electronic patient reported outcome (ePRO), toxicology lab test results, medical and pharmacy claims, medication history, FDA drug information (including the FDA-approved prescribing information for drug products), or health insurance information.

In an aspect, the pharmacological evaluation application 150 may include a fingerprint component 156 that generates a data fingerprint of collected data records for a recommendation. The system generated data ‘fingerprint’ may include auditable metadata and data keys, including, but not limited to, API's, transactions, permissions, and timestamped information of all relevant data accessed and used in any manner to create and deliver clinical decision support recommendations. The fingerprint component 156 may generate a fingerprint for each set of data records provided to the drug analysis module 160.

The drug analysis module 160 may analyze a set of data records to provide recommended treatment information. For example, the recommended treatment information may include educational materials, drug prices, medication information, or a treatment protocol. In an aspect, the treatment information may be a therapeutic success rating for a current drug and each of a plurality of alternative drugs.

In an aspect, the drug analysis module 160 may include a machine learning component 170 that manages and executes a plurality of machine learning algorithms to generate score and a weighting component 180 that applies weights based on patient preferences to determine the therapeutic success rating.

The machine learning component 170 may include shared algorithms 172 and proprietary algorithms 176. The shared algorithms 172 may be algorithms that are stored within the system 100 and may be accessed by a plurality of users. The propriety algorithms may be stored within the system 100 or may be accessible on an external system via the API 178.

In an aspect, the machine learning component 170 may provide a marketplace or exchange for machine-learning algorithms for pharmacological evaluation. The records component 154 may collect data records that are useful across a wide range of health care entities. The shared algorithms 172 may provide a set of tools that the operator of the pharmacological evaluation system 100 makes available to users. The API 174 may control access to the shared algorithms 172 such that the algorithms, models, and underlying data are protected for different users. For example, the API 174 may provide access to particular models trained for specific users. Some users may desire to utilize machine-learning algorithms that are not included within the shared algorithms 172 on the collected data records. For example, a health care payer may develop a proprietary algorithm for evaluating treatment costs under policies of the payer. The health care payer may want to execute the proprietary algorithm in association with one or more of the shared algorithms. The second API 178 may provide access to the proprietary algorithms 176. For example, the second API 178 may allow a proprietary algorithm to be loaded into the pharmacological evaluation application 150 and executed by the CPU 114. As another example, the second API 178 may facilitate a call to an external system that executes a propriety algorithm. For instance, the second API 178 may expose access to certain elements of the data records and receive results from the proprietary algorithm.

In an aspect, the machine learning component 170 (or the shared algorithms 172) may provide support for downstream machine learning algorithms (e.g., proprietary algorithms 176). For example, the machine learning component 170 may include classifiers to determine patient groupings by diagnoses, drugs used, toxicology results, user selected sources, or permissions.

In an aspect, the machine learning component 170 may coordinate various machine-learning algorithms to determine a result. The machine learning component 170 may check for consistency among machine learning algorithms. The machine learning component 170 may identify an inconsistent result between a first machine learning algorithm and a second machine learning algorithm. For example, the inconsistent result may be inclusion or exclusion from a set, or divergent scores that are typically correlated (e.g., risk scores from two different algorithms). The machine learning component 170 may identify additional decision data used by the first machine learning algorithm. The machine learning component 170 may train the second machine learning algorithm based on the additional decision data. For example, if a proprietary algorithm returns an inconsistent result such as low risk score compared to other algorithms, the machine learning component 170 may identify a factor used by the other algorithms and provide data for the other factor to the proprietary algorithm.

In an aspect, the machine learning component 170 may assist downstream machine learning algorithms. The records component 154 may gather a combination of metrics from internal sources as well as external sources. The metrics are 3 dimensional data views from the data records. The records may also be supplemented with results from other downstream machine learning algorithms. The machine learning component 170 may expose a rule set for downstream 3rd party machine learning and sample templates of rules sets as starter kits, via a user interface or template to select from a number of rule sets and dials for custom data extraction.

For example, a laboratory may develop a proprietary machine learning algorithm 176 to recommend patients for testing. In this example, the machine learning algorithm may look at daily morphine equivalent criteria to define any number of patient conditions that require alerts. The machine learning component 170 may select records and pass suggested patient populations to the machine learning algorithm. The machine learning component 170 may execute the proprietary algorithm or pass the suggested patient populations via the API 178. The proprietary algorithm may generate a medical necessity toolset that identifies tests that a patient may require. The machine learning component 170 may generate a list of patients that should get a periodic urine drug test based on a risk assessment using the medical necessity toolset.

As used herein, the term machine-learning algorithm may refer to executable code that is executed by a computer processor to process one or more elements of a data record and produce a defined result. A machine-learning algorithm may include a machine-learning model that is trained to produce the defined result. For example, the machine-learning model may include various operations and state information that reflects training of the model. Example machine-learning algorithms may include supervised learning, unsupervised learning, reinforcement learning, feature learning, sparse dictionary learning, anomaly detection, and association rules. Example models may include artificial or digital neural networks, decision trees, support vector machines, Bayesian networks, and genetic algorithms.

In an aspect, the machine learning component 170 and/or the shared algorithms 172 may include algorithms for determining a score for drug efficacy, side effect levels, and addiction risk for a current drug each of a plurality of alternative drugs. For example, the algorithms may receive the collected data records associated with a patient and a particular drug. The algorithms may each output a respective score for the patient and one of the drugs for drug efficacy, side effect levels, and addiction risk. In an aspect, the algorithms may also determine a cost score for each drug for the patient.

In an aspect, the machine learning component 170 and/or the shared algorithms 172 may include algorithms for determining a probability of a diagnoses and related disorder for a patient. For example, the disorder may be a substance use disorder. In one aspect of the invention, the substance use disorder may be an opioid use disorder. Opioid Use Disorder is a diagnosis introduced in the fifth edition of the Diagnostic and Statistical Manual of Mental Disorders, DSM-5. It combines two disorders from the previous edition of the Diagnostic and Statistical Manual, the DSM-IV-TR, known as Opioid Dependence and Opioid Abuse, and includes a wide range of illicit and prescribed drugs of the opioid class. Although the generic term, Opioid Use Disorder, is given in the DSM-5, the guidelines indicate that the actual opioid drug being used by the individual is specified in the diagnosis.

The data records may include risk metrics based on a recent monitoring window (e.g., 30 days). Example metrics may include a PDMP metric, which may be based on morphine equivalents, total prescribed opioids, presence of benzodiazepine prescriptions, total number of prescribers, and total pharmacies. A urinary drug test inconsistency may indicate discrepancies between reported drug use and detected drug use. For example, misuse (e.g., too much of a monitored drug detected in blood for written and filled prescriptions; possibly acquiring them from other sources) or diversion (too little of a monitored drug detected in blood based on written and prescriptions; possibly distributing them to other sources) are both examples of potential inconsistencies. In general, inconsistencies may be detected based on what scripts were written, what scripts were filled, who filled them and when, and what is in the patient's body—urine based on toxicology tests. Assessment metrics may include results from any assessments that the patient has previously performed, for example, for misuse risk, pain/function, depression, anxiety, and sleep. A machine-learning algorithm may include a model trained on sample patients labeled as likely OUD cases and unlikely OUD cases based on continued observation. The model may be trained to predict whether a particular patient is likely to currently experience OUD or develop OUD based on the risk metrics. The model may also be developed using reinforcement learning. The past predictions of the model may be periodically evaluated to determine accuracy, and the model may adjust weights assigned to the risk factors based on accuracy.

The weighting component 180 may weight the scores output by the machine learning algorithms based on the received patient preferences. In an aspect, the weighting component 180 may determine a therapeutic success rating. For example, the therapeutic success rating may be based on the weighted drug efficacy score, weighted side effects score, and weighted addiction risk score. In an aspect, the weighting component 180 may also determine a therapeutic index based on the therapeutic success rating and the weighted cost score.

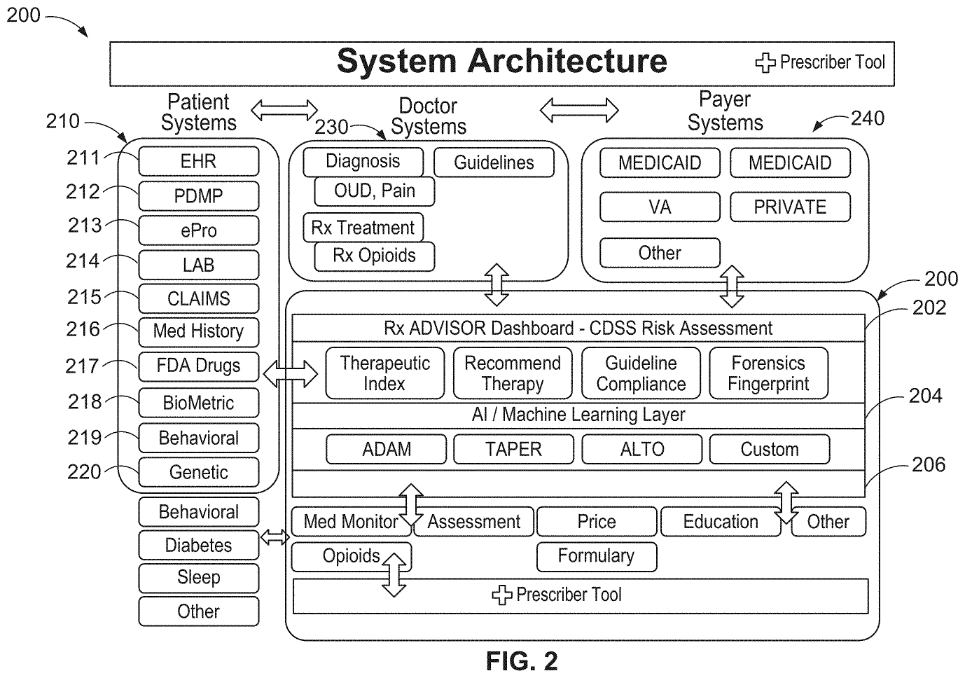

FIG. 2 illustrates an example system architecture showing interaction and data transfer between an example pharmacological evaluation system 200, patient systems 210, a doctor system 230, and payer systems 240.

The pharmacological evaluation system 200 may be an example of the pharmacological evaluation system 100. The pharmacological evaluation system 200 may include a prescription advisory dashboard 202 that provides clinical decision support services for risk assessment. The prescription advisory dashboard 202 may be an example of the patient-provider interface 152 and may provide a therapeutic index, recommended therapy, and guideline compliance for data records indicated by a forensics fingerprint. The pharmacological evaluation system 200 may include an AI/Machine Learning layer 204 that corresponds to the machine learning component 170. In an aspect, the AI/Machine Learning layer 204 may include one or more of an automated drug advice and monitoring (ADAM) system, a drug taper system (TAPER), an alternative to opioids (ALTO) system, and custom algorithms. The pharmacological evaluation system 200 may include an API layer that interacts with other systems to acquire data and/or perform specific analysis. For example, the pharmacological evaluation system 200 may utilize an external system to track drug prices and access such a system via the API layer 206. Other services that may be accessed via an API include a medication monitoring system (e.g., for opioids), an assessment system, an education system, and a formulary.

The patient systems 210 may include any system that provides information about a patient. For example, the patient systems 210 may include an electronic health record (EHR) 211, a state prescription drug monitoring program (PDMP) 212, electronic patient reported outcome (ePRO) 213, toxicology lab test results 214, medical and pharmacy claims 215, and medication history 216. In an aspect the patient systems 210 may include access to FDA drug information 217 corresponding to drugs associated with the patient or being considered for the patient. The patient systems 210 may also include information such as biometric information 218, behavioral information 219, and genetic information 220. The behavioral information 219 may include patient reported data from standardized assessment scales for clinically diagnosed conditions including, but not limited to pain, function, anxiety, depression, sleep, as recorded on scales such as PEG-3, GAD-7, PHQ-9, SOWS, COWS, etc. The behavioral information 219 may also include any information collected from a wearable device.

In an aspect, pharmacogenomics can play an important role in identifying responders and non-responders to medications, avoiding adverse events, and optimizing drug dose. The genetic information 220 may include drug labeling, which may contain information on genomic biomarkers and can describe: drug exposure and clinical response variability, risk for adverse events, genotype-specific dosing, mechanisms of drug action, polymorphic drug target and disposition genes, or trial design features. In addition, the pharmacological evaluation system 200 may access chemistry and genetics lab data for patients. Genetics labs can provide an ever increasing set of patient genetic lab data that may be used by the machine learning component 170.

The doctor systems 230 may include information provided by a health care provider. The doctor systems 230 may include a diagnosis, which may indicate opioid use disorder and/or pain, for example. The doctor systems 230 may include a prescribed treatment, which may include prescription opioids. The doctor systems 230 may include guidelines, which may define acceptable treatments for the patient.

The payer systems 240 may include any system for a payer. Example payer systems include a Medicaid system, Medicare system, veterans affairs (VA) system, private insurance, and other payers.

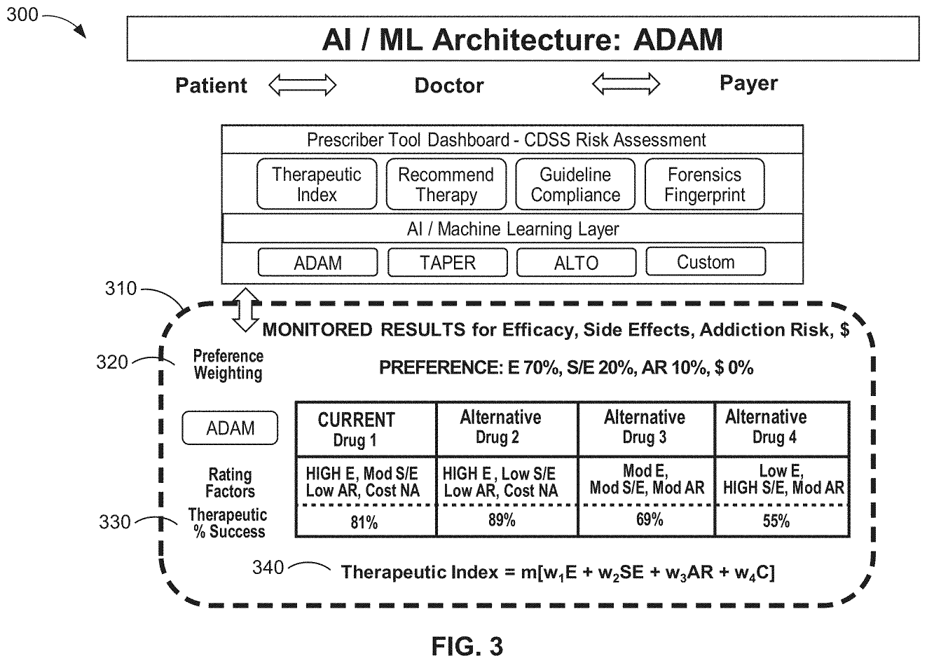

FIG. 3 is a diagram illustrating an example architecture for a drug analysis module 160. The drug analysis module 160 may include a machine learning component 170 that defines an automated drug advice and monitoring (ADAM) system 300.

The ADAM system 300 improves efficacy, cost, and quality of patient drug regimens prescribed for pain and behavioral health conditions by utilizing a clinical decision system and method driven by expanded input data sources, periodic patient feedback loops, and machine learning algorithms that are guided by user desired [prescriber and patient] weighting variables to calculate and present clinical treatment options for a patient's current drug(s), and alternative drug(s) or combinations. In short, the ADAM system presents prescribers with real-time decision support information of drug alternatives for patients to optimize the best chance of success with the lowest chance of adverse side effects and addiction risks.

The ADAM system 300 may generate monitored results 310 for efficacy, side effects, addiction risk, and cost. The ADAM system 300 may receive patient preferences via the patient-provider interface 152. The weighting component 180 may convert the patient preferences into weights 320. For example, in the illustrated scenario, efficacy may be the most important factor having a 70% weight, while side effects are assigned a 20% weight, and addiction risk is assigned a 10% weight. Cost may be assigned a 0% weight, for example, because a third party payer is covering the costs.

The ADAM system 300 may determine a score for each rating factor for the current drug and for any alternative drugs. The score may be based on baseline information for the drug, for example, according to an FDA label, published literature regarding the drug, or treatment guidelines for patients and patient demographics having a particular condition. The baseline information may be adjusted for a particular patient by applying the machine-learning algorithms. For example, the FDA label data may indicate that Drug X has 60% efficacy rate, which may be an average over population, or may be qualified by age and sex. Drug X may also have a 40% probability level of significant side effects (e.g., nausea and severe vomiting). Based on this baseline information, the ADAM system 300 may apply the patient preferences for efficacy, side effects, addiction risk and cost to adjust recommended drug choices and order the choices by therapeutic success probability. In an aspect, the ADAM system 300 may access the shared machine-learning algorithms 172, which may include a trained classifier for predicting the therapeutic success probability. For example, the classifier may be trained on patient records and results to classify a new patient record including current drug information into a predicted therapeutic success probability 330. The classifier may be continually updated using reinforcement learning to further train the classifier model based on received patient data, patient preference changes, guideline changes, and reassessment.

The ADAM system 300 may determine a therapeutic index 340. For example, the therapeutic index 340 may be calculated as m[w₁E+w₂SE+w₃AR+w₄C], where m is a matrix of patient factors, E is an efficacy rate, SE is a side effect rate, AR is an addiction risk score, and C is a cost per day. The weights, w₁-w₄, may correspond to the weights assigned by the weighting component 180 based on the patient preferences. In an aspect, the therapeutic index may be normalized and/or expressed as a percentage.

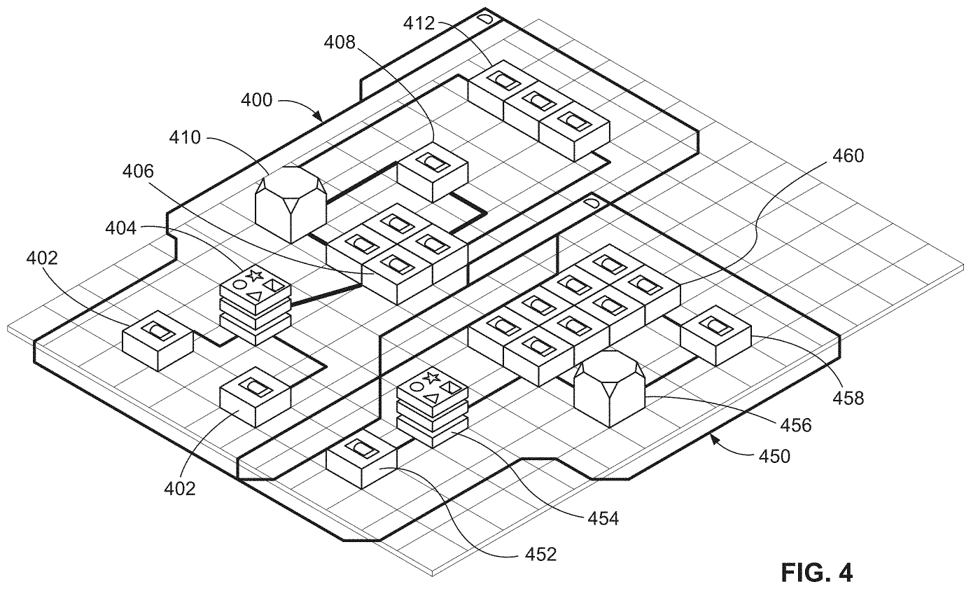

FIG. 4 illustrates an example of a network or cloud services system 400 for implementing the pharmacological evaluation system 100. The system 400 may interact with a PDMP system 450, for example, to acquire data records. The system 400 may include one or more API servers 402 that receive requests via an API. For example, the requests may be received from an application executing on a user device 120, or may be received from other servers (e.g., for doctor systems 230 or payer systems 240. An access controller 404 may verify that the request is acceptable, and add the request to worker queues 406. A scheduler 408 may send the requests to a machine-learning server array 412, which may implement the machine learning component 170. The machine-learning server array 412 may access a database 410, which may store the collected data records.

The PDMP system 450 may be an example of a patient system 210 that stores patient data. The system 400 may access the PDMP system 450 via API server 452. The PDMP system 450 may include an access controller 454, workers queues 460, a scheduler 458, and a PDMP database 456.

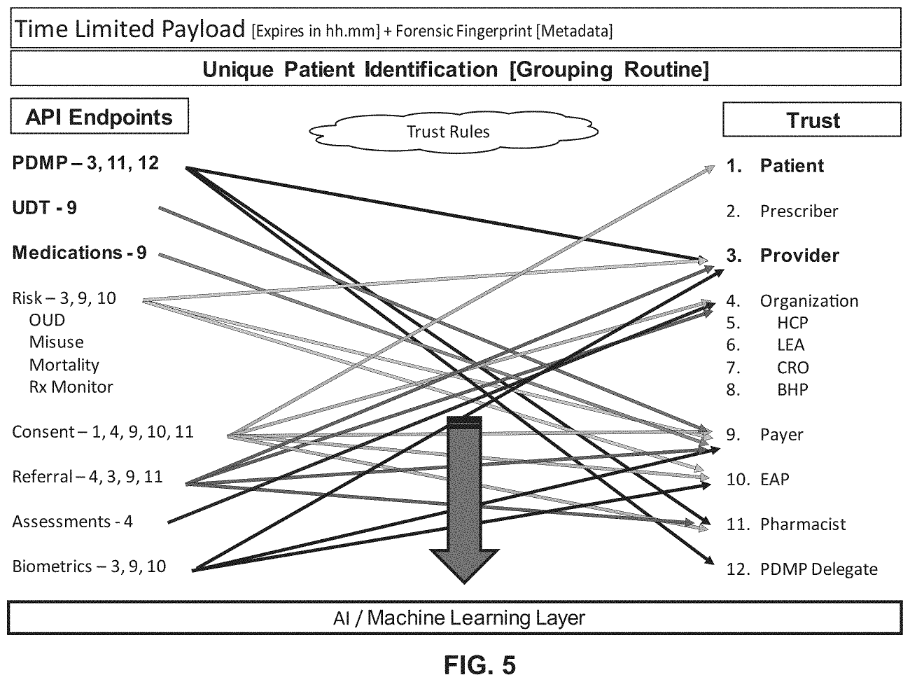

FIG. 5 illustrates an example operation of a records component 154. The records component 154 may generate a time limited payload including a plurality of data records. The time limited payload may have a set expiration time (e.g., when the underlying data is expected to be updated) and a forensic fingerprint. The forensic fingerprint may be a collection of metadata describing the source and timing of the limited time payload.

The records component 154 may identify a unique patient within the data records. For example, data records for a unique patient may use different identification numbers or names within different systems. For example, the records component 154 may identify data records with overlapping elements and conflicting elements. The records component 154 may determine, based on the overlapping elements that the data records are for the same person. The records component 154 may consolidate the data records into a single virtual data record with the conflicting elements represented by a union of the conflicting elements or the conflicting element from a data record with a highest accuracy rating.

The records component 154 may also use trust rules to control the flow of data to various entities. For example, trust rules may define an association between each data source or API endpoint and one or more trusted entities that are allowed to receive data from the API endpoint. The trust rules may be based on contractual (e.g., consent forms) or legal access to certain types of data. When a trusted entity requests an analysis (e.g., by executing one or more machine learning algorithms), the records component 154 may determine whether the data records needed for the particular request are accessible to the entity.

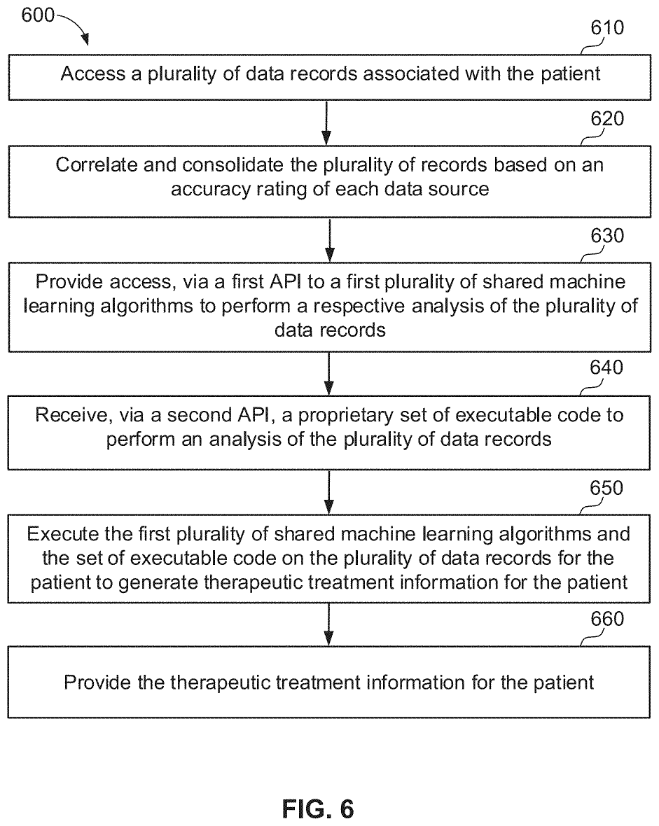

Turning to FIG. 6, an example method 600 for providing recommended therapeutic treatment information is illustrated. For example, method 600 may be performed by the pharmacological evaluation application 150 on the computer device 110.

At block 610, the method 600 may include accessing a plurality of data records associated with the patient. In an aspect, for example, the records component 154 may access a plurality of data records associated with the patient.

At block 620, the method 600 may include correlating and consolidating the plurality of records based on an accuracy rating of each data source. In an aspect, for example, the records component 154 may correlate and consolidate the plurality of records based on an accuracy rating of each data source.

At block 630, the method 600 may include providing access, via a first API to a first plurality of shared machine learning algorithms to perform a respective analysis of the plurality of data records. In an aspect, for example, the machine learning component 170 may provide access, via a first API 174 to a first plurality of shared machine learning algorithms 172 to perform a respective analysis of the plurality of data records.

At block 640, the method 600 may include receiving, via a second API, a proprietary set of executable code to perform an analysis of the plurality of data records. In an aspect, for example, the machine learning component 170 may receive, via a second API 178, a proprietary set of executable code (e.g., proprietary algorithms 176) to perform an analysis of the plurality of data records.

At block 650, the method 600 may include executing the first plurality of shared machine learning algorithms and the set of executable code on the plurality of data records for the patient to generate therapeutic treatment information for the patient. In an aspect, for example, the machine learning component 170 may execute the first plurality of shared machine learning algorithms and the set of executable code on the plurality of data records for the patient to generate therapeutic treatment information for the patient.

At block 660, the method 600 may include providing the therapeutic treatment information for the patient. In an aspect, for example, the patient-provider interface 152 may provide the therapeutic treatment information for the patient.

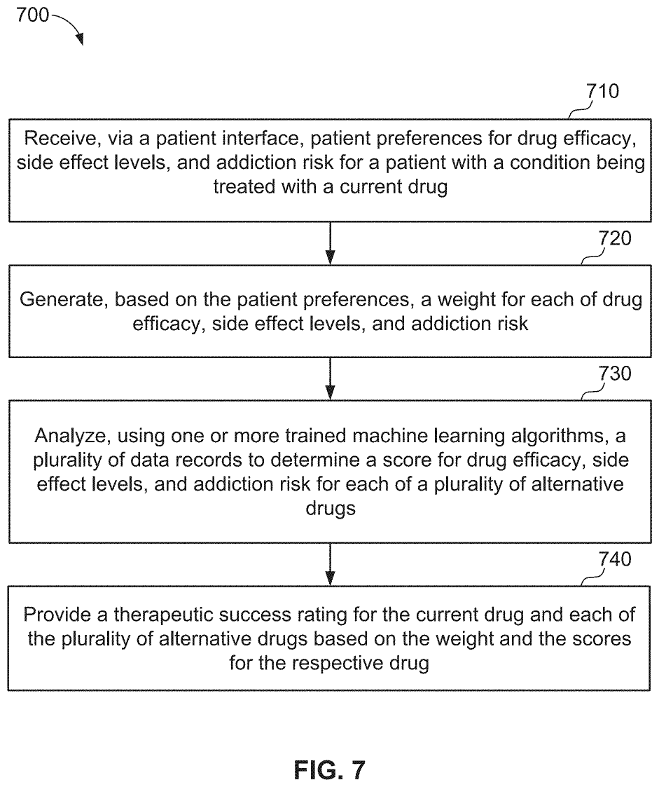

Referring now to FIG. 7, an example method 700 generates predictive probabilities of therapeutic and adverse outcomes with recommended patient treatment options for administration of drugs and drug combinations. For example, method 700 may be performed by the pharmacological evaluation application 150 on the computer device 110.

At block 710, the method 700 may include receiving, via a patient interface, patient preferences for drug efficacy, side effect levels, and addiction risk for a patient with a condition being treated with a current drug. In an aspect, for example, the patient-provider interface 152 may receive patient preferences for drug efficacy, side effect levels, and addiction risk for a patient with a condition being treated with a current drug. The patient-provider interface 152 may also receive a patient preference for cost.

At block 720, the method 700 may include generating, based on the patient preferences, a weight for each of drug efficacy, side effect levels, and addiction risk. In an aspect, for example, the weighting component 180 may generate, based on the patient preferences, a weight for each of drug efficacy, side effect levels, and addiction risk

At block 730, the method 700 may include analyzing, using one or more trained machine learning algorithms, a plurality of data records to determine a score for drug efficacy, side effect levels, and addiction risk for each of a plurality of alternative drugs. In an aspect, for example, the machine learning component 170 may analyze, using one or more trained machine learning algorithms, a plurality of data records to determine a score for drug efficacy, side effect levels, and addiction risk for each of a plurality of alternative drugs.

At block 740, the method 700 may include providing a therapeutic success rating for the current drug and each of the plurality of alternative drugs based on the weight and the scores for the respective drug. In an aspect, for example, the patient-provider interface 152 may provide a therapeutic success rating for the current drug and each of the plurality of alternative drugs based on the weight and the scores for the respective drug.

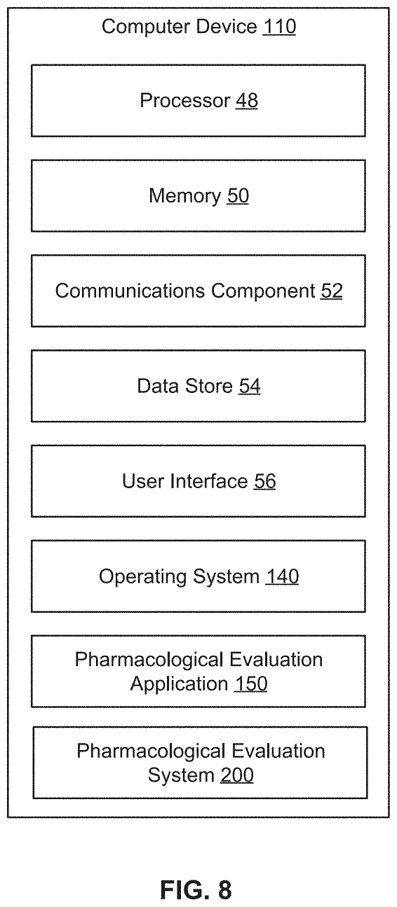

Referring now to FIG. 8, illustrated is an example computer device 110 in accordance with an implementation, including additional component details as compared to FIG. 1. In one example, computer device 110 may include processor 48 for carrying out processing functions associated with one or more of components and functions described herein. Processor 48 can include a single or multiple set of processors or multi-core processors. Moreover, processor 48 can be implemented as an integrated processing system and/or a distributed processing system. In an implementation, for example, processor 48 may include CPU 114.

In an example, computer device 110 may include memory 50 for storing instructions executable by the processor 48 for carrying out the functions described herein. In an implementation, for example, memory 50 may include memory 116. The memory 50 may include instructions for executing the pharmacological evaluation application 150.

Further, computer device 110 may include a communications component 52 that provides for establishing and maintaining communications with one or more parties utilizing hardware, software, and services as described herein. Communications component 52 may carry communications between components on computer device 110, as well as between computer device 110 and external devices, such as devices located across a communications network and/or devices serially or locally connected to computer device 110. For example, communications component 52 may include one or more buses, and may further include transmit chain components and receive chain components associated with a transmitter and receiver, respectively, operable for interfacing with external devices.

Additionally, computer device 110 may include a data store 54, which can be any suitable combination of hardware and/or software, that provides for mass storage of information, databases, and programs employed in connection with implementations described herein. For example, data store 54 may be a data repository for operating system 140 and/or applications 130. The data store may include memory 116 and/or storage device 118.

Computer device 110 may also include a user interface component 56 operable to receive inputs from a user of computer device 110 and further operable to generate outputs for presentation to the user. User interface component 56 may include one or more input devices, including but not limited to a keyboard, a number pad, a mouse, a touch-sensitive display, a digitizer, a navigation key, a function key, a microphone, a voice recognition component, any other mechanism capable of receiving an input from a user, or any combination thereof. Further, user interface component 56 may include one or more output devices, including but not limited to a display, a speaker, a haptic feedback mechanism, a printer, any other mechanism capable of presenting an output to a user, or any combination thereof.

In an implementation, user interface component 56 may transmit and/or receive messages corresponding to the operation of operating system 140 and/or applications 130. In addition, processor 48 may execute operating system 140 and/or applications 130, and memory 50 or data store 54 may store them.

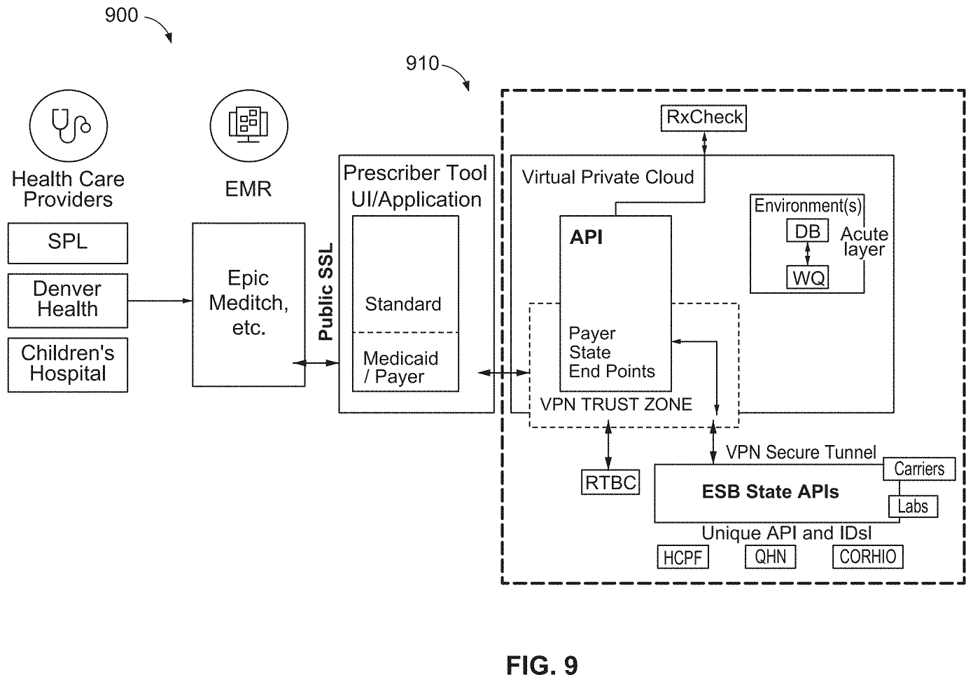

FIG. 9 is a diagram 900 illustrating an example communications architecture 910 in accordance with an implementation of the present disclosure. The communications architecture 910 establishes bidirectional data exchange for a variety of patient and medication specific data in a manner than best informs a prescriber's decision at the time of prescribing a medication. This platform is a set of services organized as functions to perform medication monitoring for controlled substances and behavioral health medications across an evolving ecosystem of connections and stakeholders. The platform integrates with EMRs and enables implementation of secure web and mobile interfaces in a highly configurable manner.

The core of the communications architecture 910 is driven by three core data streams: PDMP; toxicology; and patient provided assessments. These data streams inform the medication management analytics of the Pharmacological Evaluation system. The information is organized and served to the prescriber in digestible formats to allow for rapid decision-making based on multiple data points. Without the Pharmacological Evaluation system, prescribers have to look for the information in multiple places which takes a significant amount of time and likely prescribers are not completing the checks or are forced to make decisions with limited information.

The communications architecture 910 is a layer of technology that connects the Pharmacological Evaluation system and a variety of patient and medication specific data in a manner than best informs a prescriber's decision at the time of prescribing a medication.

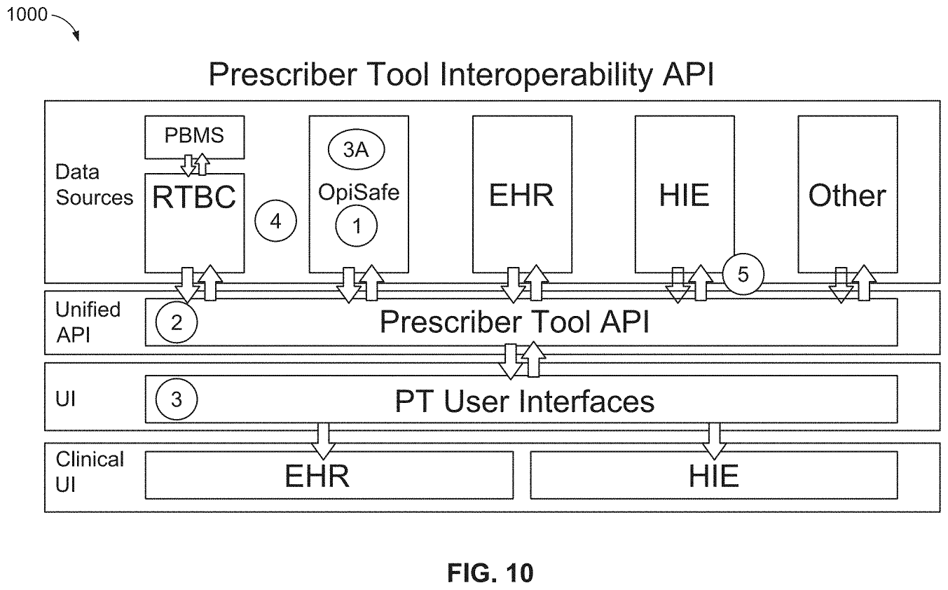

FIG. 10 is a diagram illustrating an example API structure for prescriber tool interoperability. The pharmacological evaluation system Medication Grid is incorporated into the Prescriber Tool API. The Medication Grid is unique for each Patient and binds Payer information to Patient medications with risk analysis, clinical decision support, education, side effects, therapeutic substitution and pharmacogenomics.

The pharmacological evaluation system connects via API's to Remote Benefit Checking vendors, EHRs and a variety of other data sources. API Endpoints are extended to State systems, Payers, Health Information Exchanges (HIEs) and to other services in a manner that foster faster, more pervasive adoption of the Prescriber Tool as a medication intelligence tool amongst all prescribers.

The Prescriber Tool API allows a ‘toggle’ of the Prescriber Tool UI in pharmacological evaluation system and other services, such as an RTBC integration through the Prescriber Tool API, with API endpoints delivered to the other services for connecting additional, relevant, health information. The Prescriber Tool encompasses all essential available medication information in a single user interface. By delivering an API architecture for the Prescriber Tool, all Prescribers can connect to multiple Payer RTBCs through this central infrastructure, which enables use across all Prescribers, Patients and Payers, across multiple states.

In the design of the Prescriber Tool API are permissions that allow information within the API to be connected to broader API initiatives as they evolve in the future. This increases the information flow of community data from States, to Payers and to health systems that utilize the Prescriber Tool.

The Prescriber Tool empowers Prescribers to make better prescribing decisions in real time, at the point of care. The information available for medications and patients improves upon implementation of the Prescriber Tool, and the ability of Payers and care organizations to add addition applications for their specific care delivery and outcomes goals further improves clinical decision support for medication prescribing.

The Prescriber Tool API uses SQL databases to operate and support authentication, permissions control, configuration of affiliated service API endpoint routing and logging requests to API endpoints that contain information governed by HIPAA.

The Prescriber Tool API incorporates oAuth with token-based access to API endpoints. The tokens issued by the token endpoint will incorporate permission restrictions based on where the token request originated from and the scope of access allowed at the time of the request for that service.

The Payer API endpoints are built from the ground up to include open connectivity to a variety of RTBC tools (vendor or internally developed) and management tools for the Services and Care Organizations connected to the Prescriber Tool API.

The Services API endpoints allow for applications that add value to the existing Medication Grid to connect with available medication and RTBC data to provide clinical value to the prescriber and their care organization.

The Prescribers API endpoints take existing Prescriber Tool API endpoints and adds the Payers and RTBC endpoints necessary to operate the Prescriber Tool API for these users.

The Care Organizations API endpoints take existing Prescriber Tool API endpoints and adds the Payers and RTBC endpoints necessary to operate the Prescriber Tool API for these users.

The Medical Record API endpoints that already function within the pharmacological evaluation system as connecting EHR information to the pharmacological evaluation system are added to the Prescriber Tool API to enable the HIE proxy of EHR information into the medical record API object.

Driven by the Pharmacological Evaluation Medication Grid API, machine learning algorithms and rule-based processing drive authenticated, permission based, trust rules for multiple data sources that deliver therapeutic guidelines and adversity probability charts, by drug alternatives, by prescriber and patient weighted variables.

[symbol]

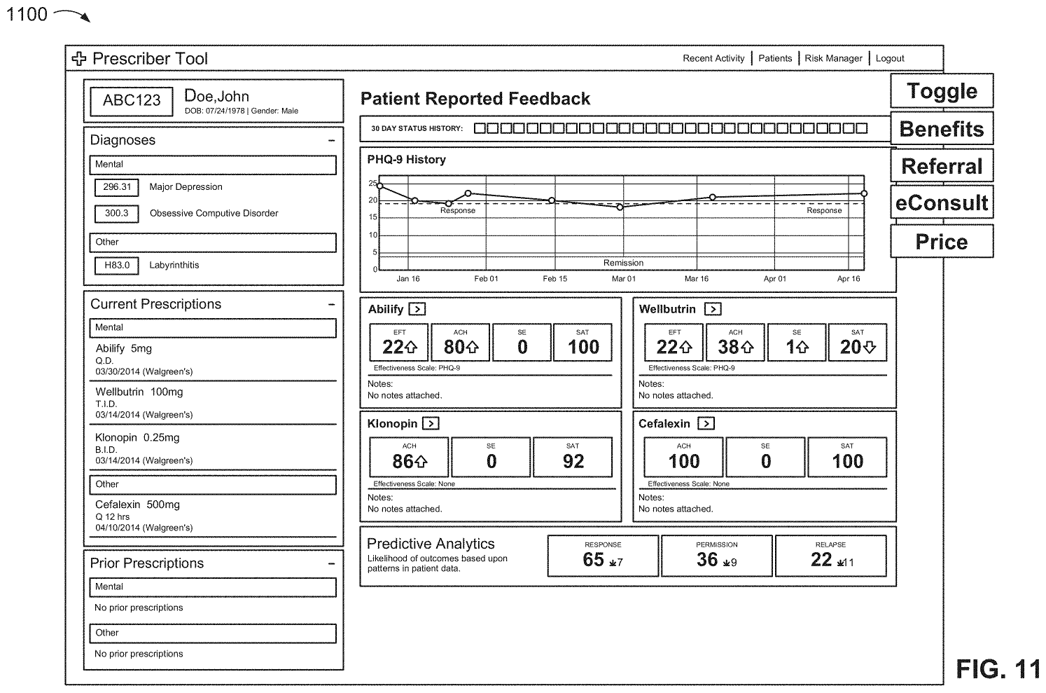

FIG. 11 is a diagram illustrating an example GUI for presenting therapeutic recommendations, in accordance with an implementation of the present disclosure. In one instance of the Prescriber Tool, a clinical view that may be presented to the Prescriber may display the current Patient drugs, compared to alternative drugs, ranked by Efficacy, Side Effects, Addiction Risk and Costs, including, but not limited to total costs and Patient cost participation. In this example, a Therapeutic Index formula is also presented to calculate the Success probability, based on the preferences set by the Prescriber and the Patient for the weighting factors.

In one instance, the Prescriber Tool is presented to a Prescriber in the form of a GUI with a Patient Medication Grid summary via a decision support dashboard, based on the Pharmacological Evaluation system. Other instances may present the information in additional forms for Prescriber interaction, recommendations, guidelines and treatment.

Based on the Pharmacological Evaluation system, the Prescriber has access to multiple data streams to assist in a better summary view for status of patients' health, condition and treatment options in near real time, and over the course of time. Having access to these data streams via the architecture is, in and of itself, a distinct and novel advantage over other electronic health record systems or applications.

Building on access to multiple data streams, the GUI conveys the information in a clinically relevant, easily understood format for the prescriber. These interfaces in effect aggregate and translate the data from many data streams into a clinical decision support tool for the prescriber that enables them to have current and complete information on the status of the patient, to better inform diagnosis and therapeutic decision making at the point of care.

Specifically, the GUI in FIG. 11 is called the Prescriber Tool Patient Medication Summary Dashboard. This interface displays information from several domains (diagnoses, current prescriptions, prior prescriptions, and patient reported feedback (data)), in summary form, color coded, with “click through” to further detail on how each piece of information was generated (i.e., the source of the data and the basis for the color coding). Thresholds are displayed for therapeutic objectives or targets (e.g., “response” or “remission” targets for the diagnosis of depression, etc.). Summaries are provided for each medication that a patient is currently receiving (displaying effectiveness, adherence, side effect, and satisfaction scores for each medication). Finally, Predictive Analytics are displayed, indicating the likelihood of specified outcomes based on patterns in the patient record, and AWL algorithms.

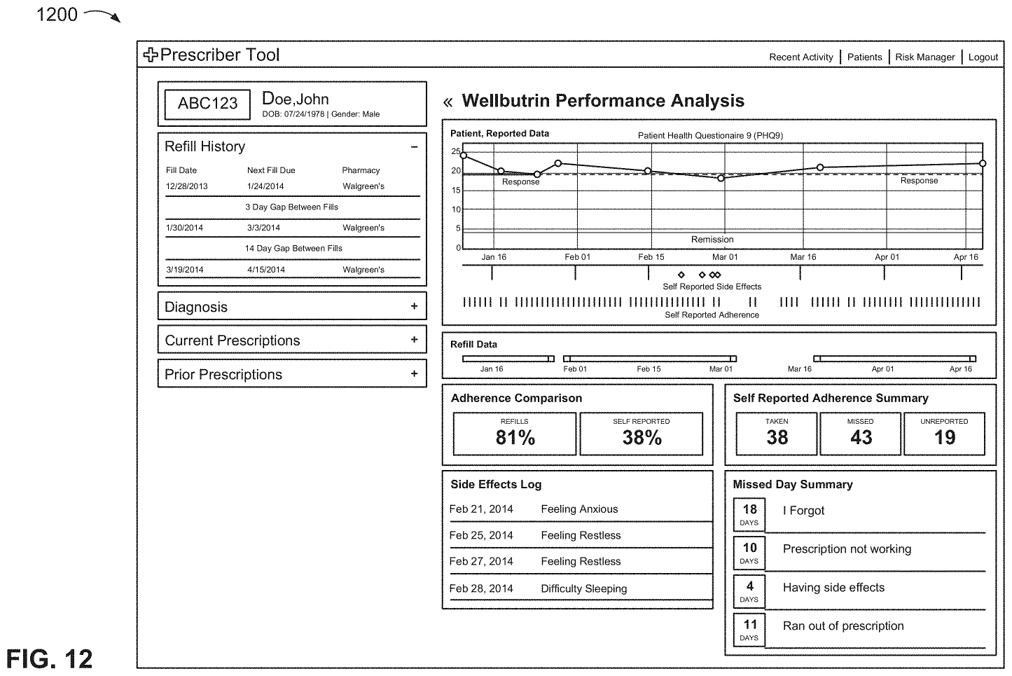

When a prescriber or other clinician “clicks” on the arrow button (“>”) next to the name of any individual medication on the Patient Medication Summary Dashboard, a second screen (interface) is displayed, called the Prescriber Tool Patient Medication Drill-Down Performance Dashboard (see FIG. 12 below). This interface displays more detailed information for the specific medication chosen by the prescriber (e.g., Wellbutrin in the example provided), including Patient Reported Data on the target outcome (e.g., PHQ-9 depression scale score), self-reported side effects, self-reported adherence, refill data, an adherence comparison (displaying refill based vs. self-reported adherence estimates), a side effects log, a self-reported adherence summary, and a missed day summary.

Lastly, the GUI 1100 includes a “navigation bar” or “Toggle” at the top right to enable prescribers to “click through” to other functionality that is useful to them in the course of prescribing or monitoring medications. These connections are driven by APIs. Examples of connections include, but are not limited to, connections to: real time benefits confirmation (RTBC) tools, real time pharmacy benefit confirmation (RTPB) tools, formulary tools, drug pricing tools, therapeutic substitution tools, patient education tools, patient monitoring tools, and other tools that may be developed or exist in the market and be relevant for prescription medication decision making.

FIG. 12 is a diagram illustrating an example GUI 1200 for presenting drug information. The GUI 1200 includes patient information, which may include: a refill history, diagnoses, current prescriptions, and prior prescriptions. The GUI 1200 may also include patient report data, refill data, an adherence comparison, a self-reported adherence summary, a side effects log, and a missed day summary.

The summary information may always be presented and flows visually left to right. Detailed information can be obtained by clicking a drill down icon for roll up data. Colors may be used to indicate potential issues: for example, colors may be: red (attention), gold (warning) and green (normal). Risks may be summarized, stacked, ranked with trends, and color coded. Guideline metrics are summarized to standards, such as CDC Opioid monitoring. Recommendations and actions are provided as drill downs.

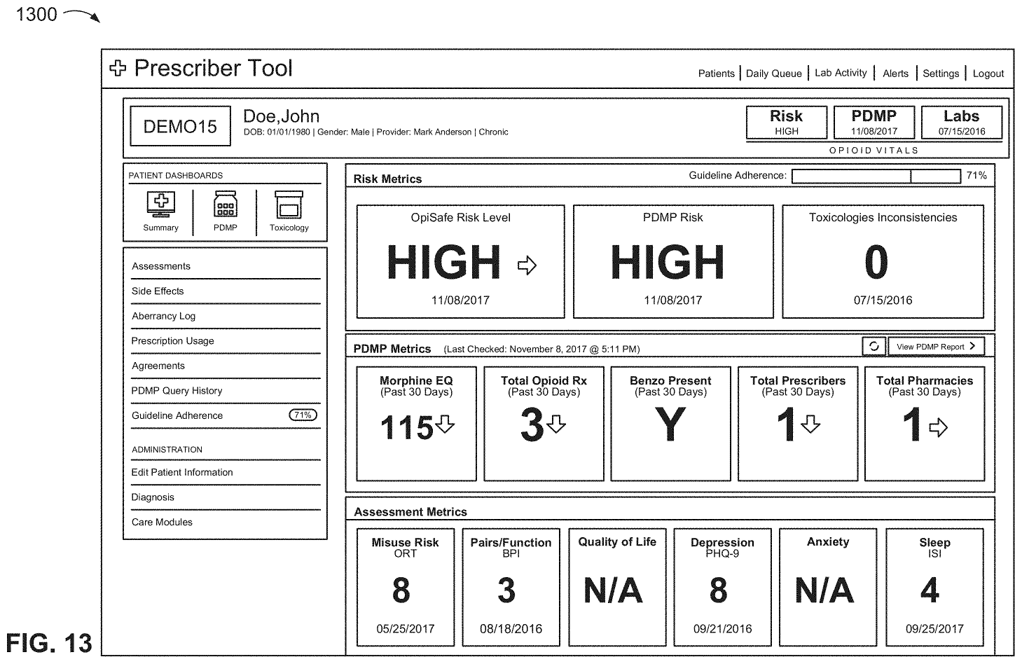

FIG. 13 is a diagram illustrating an example GUI 1300 for pharmacological evaluation. The GUI 1300 may include risk metrics including an overall risk level, a PDMP risk level, and toxicology inconsistencies. The GUI 1300 may provide details of a risk metric. For example, the PDMP metric may be based on morphine equivalents, total opioids prescribed, presence of benzodiazepine, total number of prescribers, and total pharmacies. As another example, the overall risk level may factor in a misuse risk, pain/function rating, quality of life rating, depression rating, anxiety rating, or sleep score, if available. The GUI 1300 may include a menu with options that link to further information regarding the metrics.

[symbol]

As used in this application, the terms “component,” “system” and the like are intended to include a computer-related entity, such as but not limited to hardware, firmware, a combination of hardware and software, software, or software in execution. For example, a component may be, but is not limited to being, a process running on a processor, a processor, an object, an executable, a thread of execution, a program, and/or a computer. By way of illustration, both an application running on a computer device and the computer device can be a component. One or more components can reside within a process and/or thread of execution and a component may be localized on one computer and/or distributed between two or more computers. In addition, these components can execute from various computer readable media having various data structures stored thereon. The components may communicate by way of local and/or remote processes such as in accordance with a signal having one or more data packets, such as data from one component interacting with another component in a local system, distributed system, and/or across a network such as the Internet with other systems by way of the signal.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

Various implementations or features may have been presented in terms of systems that may include a number of devices, components, modules, and the like. A person skilled in the art should understand and appreciate that the various systems may include additional devices, components, modules, etc. and/or may not include all of the devices, components, modules etc. discussed in connection with the figures. A combination of these approaches may also be used.

The various illustrative logics, logical blocks, and actions of methods described in connection with the embodiments disclosed herein may be implemented or performed with a specially-programmed one of a general purpose processor, a GPU, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but, in the alternative, the processor may be any conventional processor, controller, microcontroller, or state machine. A processor may also be implemented as a combination of computer devices, e.g., a combination of a DSP and a microprocessor, a plurality of microprocessors, one or more microprocessors in conjunction with a DSP core, or any other such configuration. Additionally, at least one processor may comprise one or more components operable to perform one or more of the steps and/or actions described above.

Further, the steps and/or actions of a method or procedure described in connection with the implementations disclosed herein may be embodied directly in hardware, in a software module executed by a processor, or in a combination of the two. A software module may reside in RAM memory, flash memory, ROM memory, EPROM memory, EEPROM memory, registers, a hard disk, a removable disk, a CD-ROM, or any other form of storage medium known in the art. An exemplary storage medium may be coupled to the processor, such that the processor can read information from, and write information to, the storage medium. In the alternative, the storage medium may be integral to the processor. Further, in some implementations, the processor and the storage medium may reside in an ASIC. Additionally, the ASIC may reside in a user terminal. In the alternative, the processor and the storage medium may reside as discrete components in a user terminal. Additionally, in some implementations, the steps and/or actions of a method or procedure may reside as one or any combination or set of codes and/or instructions on a machine readable medium and/or computer readable medium, which may be incorporated into a computer program product.

In one or more implementations, the functions described may be implemented in hardware, software, firmware, or any combination thereof. If implemented in software, the functions may be stored or transmitted as one or more instructions or code on a computer-readable medium. Computer-readable media includes both computer storage media and communication media including any medium that facilitates transfer of a computer program from one place to another. A storage medium may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes compact disc (CD), laser disc, optical disc, digital versatile disc (DVD), floppy disk and Blu-ray disc where disks usually reproduce data magnetically, while discs usually reproduce data optically with lasers. Combinations of the above should also be included within the scope of computer-readable media.

While implementations of the present disclosure have been described in connection with examples thereof, it will be understood by those skilled in the art that variations and modifications of the implementations described above may be made without departing from the scope hereof. Other implementations will be apparent to those skilled in the art from a consideration of the specification or from a practice in accordance with examples disclosed herein.

What is claimed is:

1. A method of recommending therapeutic treatment information for a patient, comprising:
accessing a plurality of data records associated with the patient;
correlating and consolidating the plurality of records based on an accuracy rating of each data source;
providing access, via a first application programming interface (API) to a first plurality of shared machine learning algorithms to perform a respective analysis of the plurality of data records;
receiving, via a second API, a proprietary set of executable code to perform an analysis of the plurality of data records;
executing the first plurality of shared machine learning algorithms and the set of executable code on the plurality of data records for the patient to generate therapeutic treatment information for the patient; and
providing the therapeutic treatment information for the patient.

2. The method of claim 1, further comprising:
identifying an inconsistent result between a first machine learning algorithm and a second machine learning algorithm;
identifying additional decision data used by the first machine learning algorithm; and
training the second machine learning algorithm based on the additional decision data.

3. The method of claim 1, wherein correlating and consolidating the data records comprises:
determining data records with overlapping elements and conflicting elements;
determining, based on the overlapping elements that the data records are for a same person; and
consolidating the data records into a single data record with the conflicting elements represented by a union of the conflicting elements or the conflicting element from a data record with a highest accuracy rating.

4. The method of claim 1, further comprising generating a forensic fingerprint of the plurality of data records including auditable data keys for records included in the plurality of data records.

5. The method of claim 4, wherein the auditable data keys identify timestamped API access, transactions, and permissions.

6. The method of claim 4, further comprising attaching the forensic fingerprint to the therapeutic treatment information.

7. A system for recommending therapeutic treatment information for a patient, comprising:
a memory storing computer-executable instructions; and
a processor configured to execute the computer-executable instructions to:
access a plurality of data records associated with the patient;
correlate and consolidate the plurality of records based on an accuracy rate of each data source;
provide access, via a first application programming interface (API) to a first plurality of shared machine learning algorithms to perform a respective analysis of the plurality of data records;
receive, via a second API, a proprietary set of executable code to perform an analysis of the plurality of data records;
execute the first plurality of shared machine learning algorithms and the set of executable code on the plurality of data records for the patient to generate therapeutic treatment information for the patient; and
provide the therapeutic treatment information for the patient.

8. The system of claim 7, wherein the processor is configured to execute the instructions to:
identify an inconsistent result between a first machine learning algorithm and a second machine learning algorithm;
identify additional decision data used by the first machine learning algorithm; and
train the second machine learning algorithm based on the additional decision data.

9. The system of claim 7, wherein the processor is configured to execute the instructions to:
determine data records with overlapping elements and conflicting elements;
determine, based on the overlapping elements that the data records are for a same person; and
consolidate the data records into a single data record with the conflicting elements represented by a union of the conflicting elements or the conflicting element from a data record with a highest accuracy rating.

10. The system of claim 7, wherein the processor is configured to execute the instructions to generate a forensic fingerprint of the plurality of data records including auditable data keys for records included in the plurality of data records.

11. The system of claim 10, wherein the auditable data keys identify timestamped API access, transactions, and permissions.

12. The system of claim 10, wherein the processor is configured to execute the instructions to attach the forensic fingerprint to the therapeutic treatment information.

13. A method of generating therapeutic recommendations and guidance for administration of drugs and drug combinations, comprising:
receiving, via a patient interface, patient preferences for drug efficacy, side effect levels, and addiction risk for a patient with a condition being treated with a current drug;
generating, based on the patient preferences, a weight for each of drug efficacy, side effect levels, and addiction risk;
analyzing, using one or more trained machine learning algorithms, a plurality of data records to determine a score for drug efficacy, side effect levels, and addiction risk for each of a plurality of alternative drugs; and
providing a therapeutic success rating for the current drug and each of the plurality of alternative drugs based on the weight and the scores for the respective drug.

14. The method of claim 13, wherein the patient preferences include a cost preference, and wherein analyzing, using one or more trained machine learning algorithms, a plurality of maintained data records to determine a score comprises determining a cost score.

15. The method of claim 14, further comprising determining a therapeutic index based on the therapeutic success rating, an adverse effects score, and the cost score.

16. The method of claim 13, further comprising:
accessing a plurality of data records associated with the patient; and
correlating and consolidating the plurality of records based on an accuracy rating of each data source;

17. The method of claim 13, wherein one or more of the trained machine-learning algorithms is trained on patient records labeled as successful or unsuccessful to predict the therapeutic success rating for the patient based on the weighted factors.

18. A system for generating therapeutic recommendations and guidance for administration of drugs and drug combinations, comprising:
a memory storing computer-executable instructions; and
a processor configured to execute the computer-executable instructions to:
receive, via a patient interface, patient preferences for drug efficacy, side effect levels, and addiction risk for a patient with a condition being treated with a current drug;
generate, based on the patient preferences, a weight for each of drug efficacy, side effect levels, and addiction risk;
analyze, using one or more trained machine learning algorithms, a plurality of data records to determine a score for drug efficacy, side effect levels, and addiction risk for each of a plurality of alternative drugs; and
provide a therapeutic success rating for the current drug and each of the plurality of alternative drugs based on the weight and the scores for the respective drug.

19. The system of claim 18, wherein the patient preferences include a cost preference, and wherein the processor is configured to execute the instructions to analyze, using one or more trained machine learning algorithms, a plurality of maintained data records to determine a cost score.

20. The system of claim 19, wherein the processor is configured to execute the instructions to determine a therapeutic index based on the therapeutic success rating, an adverse effects score, and the cost score.

21. The system of claim 18, wherein the processor is configured to execute the instructions to:
access a plurality of data records associated with the patient; and
correlate and consolidate the plurality of records based on an accuracy rating of each data source;

22. The system of claim 18, wherein one or more of the trained machine-learning algorithms is trained on patient records labeled as successful or unsuccessful to predict the therapeutic success rating for the patient based on the weighted factors.
